(19) United States
(12) Patent Application Publication
(10) Pub. No.: US 2010/0101437 A1
(43) Pub. Date: Apr. 29, 2010

(54) PRINTING MACHINE

(75) Inventor: Michael Bauernfeind, Kenzingen (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: BRUDER NEUMEISTER GMBH, Lahr (DE)

(21) Appl. No.: 12/577,325

(22) Filed: Oct. 12, 2009

(30) Foreign Application Priority Data
Oct. 24, 2008 (DE) 202008014207.5

(51) Int. Cl. A01H 5/02

(52) U.S. Cl. 101/171

(57) ABSTRACT

A printing machine for multi-color printing, including at least one camera (6) for monitoring a conformity of different ink applications. The camera (7) in arranged in the printing machine (1) via a holder (7), and a clear cover (9) for an optic (10) of the camera provides protection from contamination or ink splatter. The cover (9) is embodied as a strip (11) of clear film, which can be moved along a direction of extension, with a width of the strip being at least equivalent to a width of at least one of the optic (10) or a measuring field of the camera (6) detected therewith. A portion (12) of the strip (11) is arranged in a radiation path in front of the optic (10) of the camera (6), and the portion (12) of the strip (11) located in the radiation path in front of the optic is moved constantly or from time to time longitudinally in reference to the direction of extension and in reference to the camera (6), so that a used portion of the strip (11) can be brought out of the radiation path of the optic and an unused portion of the strip can be brought into the radiation path of the optic.

### CROSS-REFERENCE TO RELATED APPLICATIONS

This application claims the benefit of German Patent Application No. 20 2008 014 207.5, filed Oct. 24, 2008, which is incorporated herein by reference as if fully set forth.

### BACKGROUND

The invention relates to a printing machine for multi-color printing having at least one camera for monitoring the conformity of different ink applications, with the camera arranged in the printing machine via a holder comprising a clear cover of its optic as a protection from contamination or ink splatter.

Such printing machines and particularly the corresponding cameras are known and used, for example, in the automatic readjustment of the positioning of the printing rollers on the commodity to be printed. Here, the camera is generally arranged in the area of the printing machine, in which the printed commodity, particularly paper, and the ink located thereupon have not yet dried so that ink splatter or other contaminants of the optic of the camera can appear which compromises its functionality.

In the past, such cameras had to be cleaned from time to time, and in order to avoid said necessity it has become known to cover the optic of the camera by exchangeable glass panes. This way, the glass pane can be exchanged from time to time, avoiding an extended interruption for a cleaning process. Although such an exchange of the exchangeable glass pane requires an interruption of the operation of the printing machine shorter than the period necessary for a complete cleaning of the optic of the camera, each interruption represents an undesired disturbance in the operation, though.

### SUMMARY

Therefore, the object is to provide a printing machine with a camera of the type mentioned at the outset, in which an interruption of the printing operation for removing contaminations present on the cover of the camera optic is avoided.

In order to attain this object, it is provided that in a printing machine of the type mentioned at the outset the cover is embodied as a strip comprises a clear film that is movable along its direction of extension and with its width being at least equivalent to the width of the optic and/or the measured area detected by said camera, that a portion of the strip is arranged in the radiation path in front of the optic of the camera, and that the portion of the strip located in the radiation path in front of the optic is always or from time to time can travel in reference to the camera along its direction of extension, thus a used portion of the strip can be moved out of the radiation path of the optic and an unused portion of the strip can be moved into the radiation path of the optic.

Via subsequent imaging software it is possible to limit the image detected by the camera to a partial area, in which for example edge regions of the detected image are blinded out by way of software. In this case, it is already sufficient for the strip to cover the area equivalent to the further processed partial area of the detected image and thus the extension of the measuring field of the camera.

This way, it is possible to shield the optic of the camera with a very inexpensive material, namely a clear film, and to ensure, depending on a certain level of contamination or depending on additional processing parameters or by a preset continuous traveling, that always sufficient clarity of said shield is given. This way, the necessity is avoided to directly clean the camera optic or to exchange its covering pane from time to time and thus also the need for interruptions.

ere, it is beneficial for the unused portion of the strip to be stored on a roller, from which the strip can be rolled off into the radiation path in front of the optic. This way, a relatively long cover strip can be provided, which can be used for an appropriate length of time. For a simple and useful handling when feeding said strip past the camera the used portion of the strip can be wound onto a spool or a drum. In particular, the spool or drum receiving the used portion of the strip can be motor-driven. This way, the entire process can be automated.

In particular for feeding the cover film or the cover strip from time to time the motorized drive of the spool or drum receiving the used portion of the strip may comprise a control.

The control can be adjusted to trigger the operation of said motorized drive in predetermined intervals and/or after a predetermined amount of the printed commodity has passed, with the intervals or the predetermined amount of the printed commodity is set such that a predetermined level of contamination at the film not compromising the function of the camera is not exceeded.

Alternatively or additionally a sensor may be provided, by which the level of contamination of the portion of the strip is monitored, located in the radiation path in front of the optic, with the control being set to trigger the operation of the motorized drive when a predetermined level of contamination is reached. This way, a particularly constant and material-saving use of the strip is achieved, and any interference of the function of the camera by the covering film becoming turbid in the radiation path is avoided.

Here, it is particularly beneficial when a sensor serves to interpret the image of the camera, particularly via a CCD-chip or a detector field. Said image interpretation is already in use for detecting color positioning markers or the general monitoring of the conformity of the different ink applications and therefore it can also be used with little expense for detecting the level of contamination.

It is beneficial, here, when the roller storing the unused portion of the strip is supported on a holder, which can be mounted at the camera near the optic, with the rotary axis of the roller being arranged perpendicularly in reference to the feed direction of the strip. Preferably, the holder can be mounted in a detachable fashion, facilitating access for exchanging the roller.

The spool or drum receiving the used portion of the strip can also be mounted at the camera via a holder, preferably in a detachable fashion. This way it is also easy for the user to exchange these rollers including the respective spool or drums after the film has been consumed or to reinstall them after providing them with a fresh film, if applicable.

The arrangement can be operated in a particularly economical fashion, when the roller storing the unused portion of the strip is arranged on a spool or a drum, which can be inserted as the spool or drum receiving the used portion of the next strip after the strip has been consumed. This way, after the strip of the film has been consumed, the original storage roller can be used as the receiving roller for the next strip of film.

The holders for the unused portion of the roller storing the strip, i.e. the storage roller, and the spool or drum receiving the used portion of the strip, i.e. the receiving roller, can be connected to each other, further facilitating the exchange of a used film. Here, the rollers can be mounted at this holder in a detachable fashion, in order to allow the use of the original storage roller as a receiving roller in this case, too.

A guide can be embodied at the camera, matching a counter-guide of the holders for the roller storing the unused portion of the strip and for the spool or drum receiving the used portion of the strip. In particular, this guide can be aligned perpendicular in reference to the direction of feed of said strip. By pairing the guide and the counter guide the holders embodied as a compound part or separate parts can easily be attached and/or inserted, rendering the time necessary for changing the film rollers to be respectively short.

For example, at least one partially open recess may be provided as the guide, preferably two recesses opposite each other in reference to the radiation path of the optic and/or extending parallel in reference to each other, into which the counter guide of the holders can be inserted. This way, the guide to receive the joint holder fits the rollers, and the rollers with their holders can be exchanged in their entirety like a module.

In order to largely avoid any optic distortions in the radiation path, which might be caused by the strip and which could compromise the monitoring for conformity of the different ink applications, a defined guide of the strip is advantageous where the radiation beam of the optic is passed. This may be achieved, for example, via guiding elements such as rollers or gliding pins or gliding edges. However, this is particularly simple when in the operating position the strip contacts the front-most element of the optic in the radiation path of the optic, particularly a lens or a glass pane that is a part of the camera. In particular, it may be provided for the strip to be contacting said element in a planar fashion.

Here, it is particularly beneficial for the strip to contact the optic of the camera under stress and to glide along it when traveling. This can be achieved, for example, by arranging the frontal limit of the optic in the radiation path in front of a virtual tangential line in the radiation path, which contacts the roller storing the unused portion of the strip and the spool or drum receiving the used portion of the strip.

In practice, this is advantageously achieved in the tangential plane contacting the largest diameter of the roller storing the unused portion of the strip and the spool or drum receiving the used portion of the strip.

A compact design results when the motor drive is connected to the holders for the roller storing the unused portion of the strip and the spool or drum receiving the used portion of the strip.

For example, it may be provided that electric connection means for the motor drive are connected to the holders, that electric counter connection means are provided at the camera, and that when mounting the holders at the camera an electric contact is generated between the electric connection means and the electric counter connection means to operate the motor drive. This way, the motor drive can be electrically connected when the counter guide is completely inserted into the guide at the camera. This way, an automatically functioning module is provided, which is mounted to provide protection from contamination at the camera and which can be exchanged after the strip has been consumed.

### BRIEF DESCRIPTION OF THE DRAWINGS

The invention is now explained in greater detail using an exemplary embodiment; however, it is not limited to this exemplary embodiment. Further exemplary embodiments result from combining the features of the exemplary embodiment with each other and/or with the features of the claims and/or by using the described invention in other types of printing machines.

Shown are:

FIG. 1 is a schematic view of a printing machine,

FIG. 2 is a detailed view of the camera of FIG. 1 with the cover being removed, and

FIG. 3 is an exploded illustration of the insertion module with the cover of FIG. 2.

### DETAILED DESCRIPTION OF THE PREFERRED EMBODIMENTS

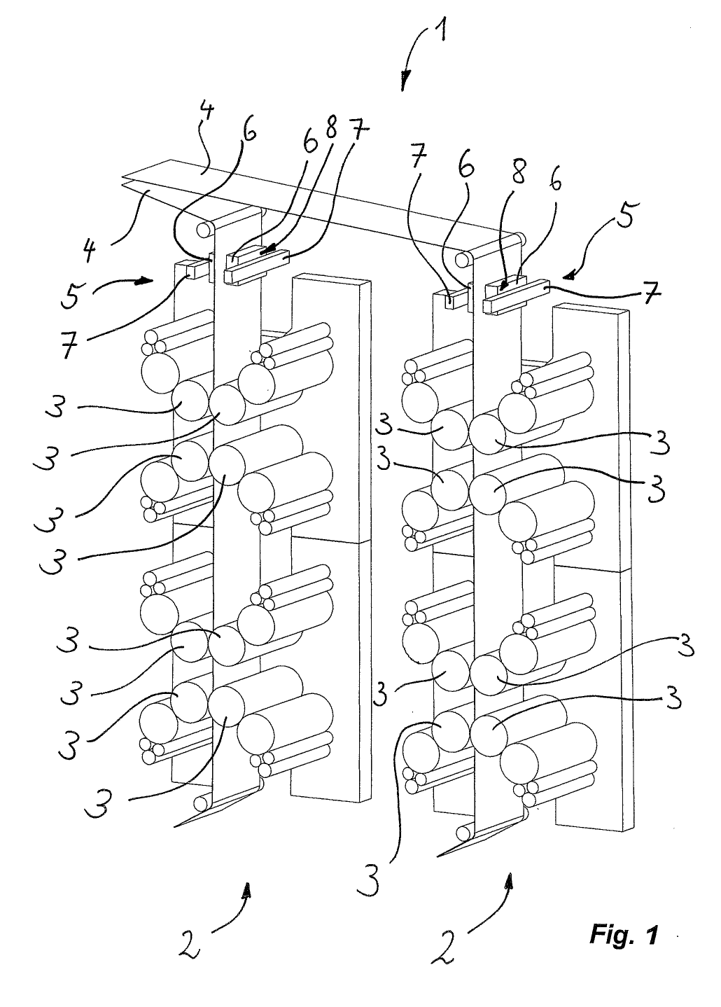

The printing machine, shown schematically in FIG. 1 and marked 1 in its entirety, has two printing towers 2, with eight printing rollers 3 each being provided for the multi-color printing of a paper web on both sides, which is intended as the commodity to be printed.

The paper web 4 is respectively guided through the printing towers 2 from the bottom towards the top. In other types of printing machines the progression of the paper web 4 may deviate from the progression shown.

Cameras 6 are arranged at the outlet 5 of the paper webs 4 out of the printing towers 2, by which the conformity of the different ink applications onto the paper webs 4 is monitored. This occurs by recognizing and interpreting, for example, colored markers printed at the edge of the paper webs or by evaluating the printed image.

Each camera 6 is provided to monitor one side of the paper web 4, with the optic of the camera 6 intended to recognize the printed image or the color markers facing the side of the paper web respectively to be monitored.

The cameras 6 are arranged in the printing machine 1 via holders 7, with said holders 7 being embodied as rails aligned perpendicular in reference to the traveling direction of the paper web 4, and the rear 8 of the cameras 7 being fastened at said rails.

After the paper webs 4 have left the outlets 5, they are fed to further processing, particularly to a cutting or folding arrangement, not shown in greater detail.

The monitoring of the conformity of the different ink applications on the paper web 4 is used to control the positioning of the printing rollers 3, in order to this way achieve a multi-color print of high quality.

In order to form a control path with reaction times as short as possible each camera 6 must be arranged as close as possible behind the last printing roller 3 in the travel direction of the paper web 4. This requires that the camera 6 is subject to contamination by the ink that is not yet completely dry.

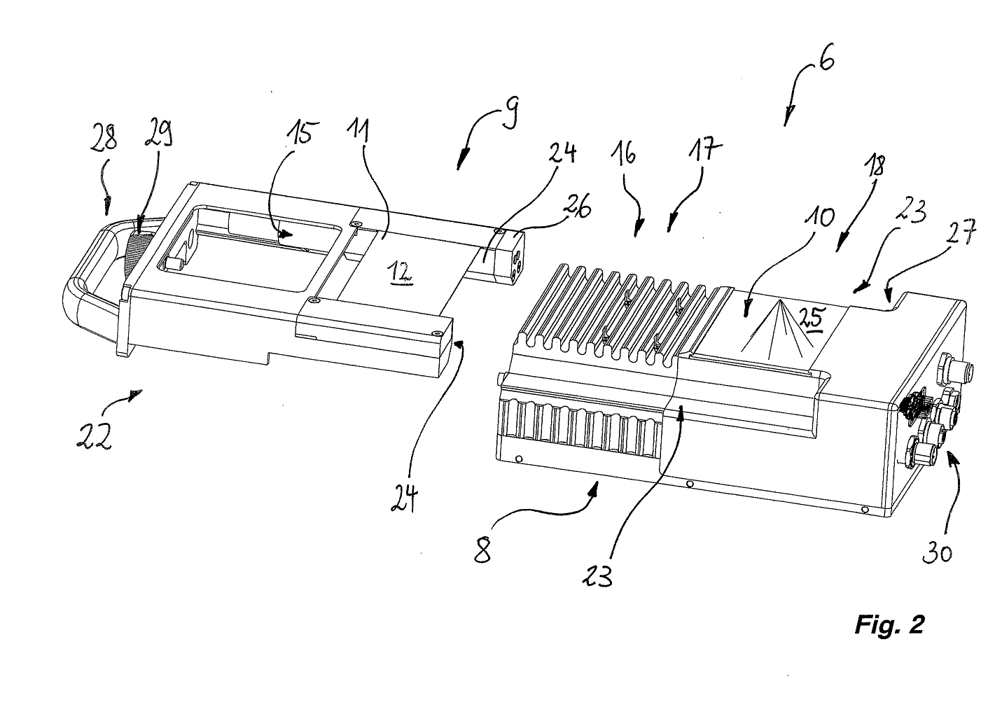

As a protection from said ink splatter and general contamination the camera 6 comprises a cover 9, which is shown in greater detail in FIG. 2.

FIG. 2 shows a camera 6 in a perspective view, in which the rear 8 of the camera 6 is arranged oriented downwards.

The cover 9 is embodied as a strip 11 comprising a clear film, thus a cover strip. The width of the strip 11 is not smaller than the width of the optic 10 and/or the measuring field detected by the camera 6. When the cover 9 is arranged at the camera 6 such that a partial section 12 of the strip 11 covers the optic 10, said optic 10 is protected from contamination.

The strip 11 can be moved along its direction of extension, allowing a consumed portion of the strip 11, i.e. covered with ink splatter and/or other contaminants, to be brought out of the radiation path of the optic 10. Here, simultaneously an unused portion of the strip 11, i.e. not yet showing any contaminants, enters the radiation path of the optic 10.

This process occurs from time to time as needed or constantly at a predetermined feeding speed.

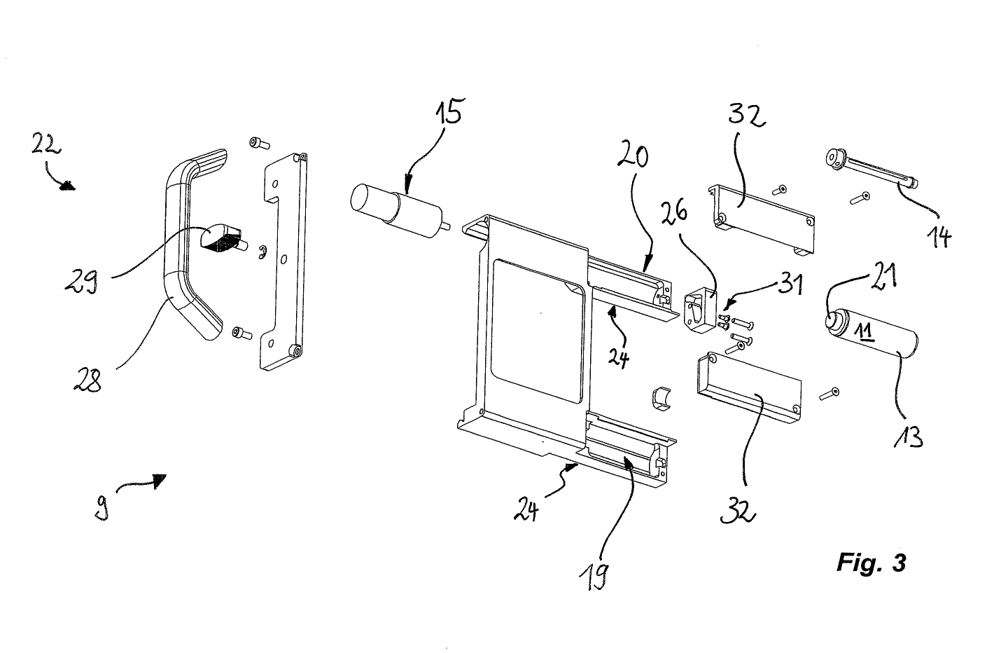

The unused portion of the strip 11 is provided on a roller 13, clearly discernible in FIG. 3, from which it can be rolled off as needed and can be brought into the radiation path in front of the optic 10.

The used portion of the strip 11 is wound onto a spool 14, also discernible in FIG. 3.

In order to provide the feed, thus the further traveling of the strip 11, the spool 14 may be motor driven. Here, one end of the strip 11 is fixed at the spool 14 so that the driven spool 14 winds up the strip 11.

The spool 14 is driven by an electric drive 15. For this purpose the camera 6 comprises an integrated PC or a calculator, which provides, among other things, the control 16 for operating the drive 15. The integrated PC, set to interpret the printed image and/or the color markers detected by the optic 10, comprises image processing, established for the image interpretation 17 reading the CCD-chip 18 provided as the detector field in the optic 10 and by which in the printed image and/or the color markers deviations from a sample can be detected using the data collected, and a processing electronic by which control signals can be generated from the detected deviations for positioning the printing rollers 3 in the printing towers 2 in reference to the paper webs 4.

The contamination of the portion 12 of the strip 11 located in front of the optic 10 can also be determined from the data read by the CCD-chip 18. Thus, a sensor is embodied to monitor the level of contamination of the strip 11, and the control 16 can operate the motor drive 15 when a predetermined level of contamination has been reached, at which the uncompromised detection of the printed image and/or the color markers is no longer possible.

As discernible in FIG. 3, the roller 13 storing the unused portion of the strip 11 is supported rotationally for the use in a holder 19. in the operating position of the cover 9, the holder 19 is arranged in the proximity of the optic 10 and causes an alignment of the rotary axis of the roller 13 perpendicular in reference to the direction of feed of said strip 11 and parallel in reference to the virtual plane resulting from the portion 12 of the strip 11 arranged in front of the optic 10.

The used portion of the strip 11 is wound onto the spool 14 discernible in FIG. 3. In the operating position the spool 14 is also arranged in a holder 20 and supported in a rotational fashion.

The roller 13 is wound onto a spool 21, which is shaped identical to the spool 14 and thus can be inserted into the holder 19 as well as the holder 20. Thus, after the unused strip 11 has been completely wound off the roller 13, the spool 21 can be used in the holder 20 to accept the used portion of a next strip 11.

The holders 19, 20 can each be closed by a lid 32 in order to protect the portion of the strip 11 wound onto the spool 14, 21 from contamination.

The holder 19 is connected to the holder 20. Therefore the two holders 19, 20 form parts of an insertion module 22 supporting the cover 9.

Two parallel recesses 23 are formed at the camera 6 as guides for the insertion module 22, into which a matching counter guide 24 can be inserted to bring the insertion module 22 into its operation position in which the cover 9 covers the optic 10. The counter guide 24 is embodied in form of two legs, with each leg forming a holder 19, 20 and thus accepting the roller 13 and/or the spool 14.

The optic 10 of the camera 6 is closed by a glass pane 25, which forms the front-most element in the radiation path of the optic 10. The radiation path of the optic 10 extends from the paper web 4 to the CCD-chip 18 of the camera 6.

The portion 12 of the strip 11 forming the cover 9 of the optic 10 contacts the glass pane 25 and is supported on the glass pane 25 in a planar fashion. This is achieved such that, by the arrangement of the spools 14, 21 in their holders 20, 19 and the geometric dimensions of the legs of the counter guide 24 and the recesses 23, the virtual plane, defined by the outside of the glass pane 25, is a tangential plane at the largest diameter of the roller 13 storing the unused portion of the strip 11, ergo the diameter of the fully wound spool 21, and the largest diameter of the spool 14 receiving the used portion of the strip 11, ergo the diameter of the fully wound spool 14.

This way, the strip 11 is pressed to the glass pane 25 and contacts it under stress when the strip 11 is subjected to a tension by impinging the spools 14 and 21 of the strip 11 in the opposite direction. This impingement is achieved by inhibiting the rotary motion of the spool 21 and by the motor drive 15 for the coil 14. The strip 11 then glides along the glass pane 25 during its further travel or its feed.

The motor drive 15 is also arranged at the insertion module 22, so that the drive 15 can be connected to the holders 19, 20 and can be used with them when the counter guide 24 is inserted into the recesses 23 in the operational position.

An electric connection means 26 with electric contact pins 31 discernible in FIG. 3 is arranged at one leg of the counter guide 24 and can be brought into an effective contact to an electric counter connection means 27 arranged in a recess 23 when inserted into the recess 23. This way, an electric connection is made of the motor drive 15 to the control 16.

In order to facilitate the removal and mounting of the insertion module 22, the module comprises a handle 28. A rotational lock 29 prevents any unintended loosening of the insertion module 22 out of its operating position. The direction of insertion of the insertion module 22, predetermined by the recesses 23 and the counter guides 24, is aligned parallel in reference to the direction of extension of the holders 7 in FIG. 1.

At the rear side of the insertion module 22 in reference to the direction of insertion, connections 30 are provided at the camera 6 to exchange data and supply the camera 6 with electric energy.

A cover strip 11 for the optic 10 of a camera 6 provided to monitor the conformity of the ink applications is provided in the printing machine 1 for multi-colored printing, by which the optic 10 is protected from contamination by ink splatter. The cover strip 11 is stored on a roller 13 and is guided past the optic 10 and wound onto a receiving spool 14. The feeding motion of the cover strip 11 past the optic 10 can be determined by the level of contamination of the cover strip 11, which can be detected by the camera 6 and an image interpreting 17 connected thereto, or by operating parameters.

1. A printing machine for multi-color printing, comprising at least one camera (6) for monitoring a conformity of different ink applications, the camera (7) in arranged in the printing machine (1) via a holder (7) comprising a clear cover (9) for an optic (10) of the camera as a protection from contamination or ink splatter, the cover (9) is embodied as a strip (11) comprising a clear film, which can be moved along a direction of extension thereof and with a width of the strip being at least equivalent to a width of at least one of the optic (10) or a measuring field of the camera (6) detected therewith, a portion (12) of the strip (11) is arranged in a radiation path in front of the optic (10) of the camera (6), and the portion (12) of the strip (11) located in the radiation path in front of the optic is moved constantly or from time to time longitudinally in reference to the direction of extension and in reference to the camera (6), so that a used portion of the strip (11) can be brought out of the radiation path of the optic and an unused portion of the strip can be brought into the radiation path of the optic.

2. A printing machine according to claim 1, wherein the unused portion of the strip (11) is stored on a roller (13), from which the strip (11) can be rolled off in the radiation path in front of the optic (10).

3. A printing machine according to claim 1, wherein the used portion of the strip (11) can be wound onto a spool (14) or a drum.

4. A printing machine according to claim 1, wherein the spool (13) or the drum receiving the used portion of the strip (11) is motor driven by a motor drive.

5. A printing machine according to claim 4, wherein the motor drive (15) of the spool (14) or the drum receiving the used portion of the strip (11) comprises a control (16).

6. A printing machine according to claim 5, wherein the control (16) is provided to trigger an operation of the motor drive (15) in at least one of predetermined intervals or after a predetermined amount of a printed commodity (4) has passed.

7. A printing machine according to claim 5, wherein a sensor is embodied by which a level of contamination of the portion (12) of the strip (11) located in the radiation path in front of the optic (10) can be monitored and the control (16) is provided to trigger the operation of the motor drive when a predetermined level of contamination is reached.

8. A printing machine according to claim 7, wherein image interpretation (17) of the camera (6) serves as the sensor.

9. A printing machine according to claim 2, wherein the roller (13) storing the unused portion of the strip (11) is supported by a holder (19) that can be mounted to the camera (6) in proximity to the optic (10) in a detachable fashion, with a rotary axis of the roller (13) being arranged perpendicular in reference to a direction of feed of the strip (11).

10. A printing machine according to claim 9, wherein a spool (14) or the drum receiving the used portion of the strip (11) is mounted via a holder (20) at the camera (6) in a detachable fashion.

11. A printing machine according to claim 9, wherein the roller (13) storing the unused portion of the strip (11) is arranged on a spool (21) or a drum, which after the strip (11) has been consumed can be inserted as the spool (14) or drum receiving the used portion of a next one of the strips (11).

12. A printing machine according to claim 2, wherein the roller (13) storing the unused portion of the strip (11) and a spool (14) or drum receiving the used portion of the strip (11) are mounted in respective holders (19, 20) in a detachable fashion.

13. A printing machine according to claim 12, wherein the holders (19, 20) for the roller (13) storing the unused portion of the strip (11) and for the spool (14) or drum receiving the used portion of the strip (11) are connected to each other.

14. A printing machine according to claim 13, wherein a guide (23) is embodied at the camera (6), aligned perpendicularly in reference to a direction of feed of the strip (11), and matches a counter guide (24) of the holders (19, 20) for the roller (13) storing the unused portion of the strip (11) and for the spool (14) or drum receiving the used portion of the strip (11).

15. A printing machine according to claim 14, wherein the guide (23) at the camera (6) comprises at least one partially open recess into which the counter guide (24) of the holders (19, 20) can be inserted.

16. A printing machine according to claim 1, wherein in the operating position the strip (11) contacts a front-most element (25) of the optic (10).

17. A printing machine according to claim 16, wherein the strip (11) contacts the optic (10) of the camera (6) under stress and glides along it when further moved.

18. A printing machine according to claim 16, wherein the front-most element (25) of the optic (6) in the radiation path is arranged in front of a virtual tangential plane in the radiation path, which contacts a roller (13) storing the unused portion of the strip (11) and a spool (14) or drum receiving the used portion of the strip (11).

19. A printing machine according to claim 18, wherein the tangential plane contacts a largest diameter of the roller (13) storing the unused portion of the strip (11) and the spool (14) or drum receiving the used portion of the strip (11).

20. A printing machine according to claim 12, wherein a motor drive (15) is connected to the holders (19, 20) for the roller (13) storing the unused portion of the strip (11) and a spool (14) or drum receiving the used portion of the strip (11).

21. A printing machine according to claim 20, wherein an electric connection (26) for the motor drive (15) are connected to the holders (19, 20), an electric counter connection (27) is provided at the camera (6), and in the operating position of the holders (19, 20) an electric contact to operate the motor drive (15) is generated at the camera (6) between the electric connection (26) and the electric counter connection (27).
